(19) United States
(12) Patent Application Publication
(10) Pub. No.: US 2014/0348921 A1
(43) Pub. Date: Nov. 27, 2014

(54) NOVEL HEMOSTATIC COMPOSITION

(71) Applicant: Patrick Lesage, Saint Malo (FR)

(72) Inventor: Patrick Lesage, Saint Malo (FR)

(21) Appl. No.: 14/233,673

(22) PCT Filed: Jul. 18, 2012
(86) PCT No.: PCT/FR2012/051700

§ 371 Date: Aug. 4, 2014

(30) Foreign Application Priority Data
Jul. 20, 2011 (FR) 1156584

(51) Int. Cl. A61K 33/06; A61K 31/167; A61K 33/26

(52) U.S. Cl. CPC A61K 33/06; A61K 33/26; A61K 31/167
USPC 424/489; 424/685; 424/646

(57) ABSTRACT

The present invention relates to a novel hemostatic composition notably useful to ensure good hemostasis and maintain the sulcular opening after a gingival eviction procedure.

According to the invention, this composition is in the form of an extrudable hydrophilic paste and contains, expressed as percentage by weight relative to the total weight of said composition:
between 5 and 15% of an astringent, preferably aluminum chloride;
between 2 and 15% of kaolinitic clay, preferably kaolin;
between 10 and 20% of a texturizing agent comprising one (or more) naturally-occurring polysaccharide(s) with gelling or thickening power optionally combined with plant fibers;
between 50 and 70% of water;
between 0 and 20% of a humectant;
   the total quantity of water and humectant being comprised between 55 and 75% by weight relative to the total weight of the composition.

The present invention relates to a novel hemostatic composition notably useful to ensure good hemostasis and maintain the sulcular opening after a gingival eviction procedure. It is applied in the dental field.

The gingival crevice is a virtual space, located between the tooth and the gum, which it is necessary to widen and dry and/or keep open and dry in order to carry out various dental care procedures, for example prior to taking an impression.

Generally, widening of the gingival crevice can be obtained:
either by eviction or gingivectomy, in particular in the case of a thick marginal gingiva, for example using a laser, an electric bistoury or a diamond charged drill;
or by retraction, in particular in the case of a thin or medium marginal gingiva, by inserting into the gingival crevice a preformed material, such as cotton cord optionally impregnated with a solution intended to promote retraction, or an injectable composition capable of hardening by chemical reaction or by physical swelling.

Patent EP 0 477 244 describes an insertion material intended to widen the gingival crevice by retraction of original design which is in the form of a biocompatible and extrudable paste comprising essentially:
an agent intended to absorb fluids, such as in particular kaolin clay (or China clay) in a quantity of 65 to 70% by weight;
water in a quantity of 24 to 27% by weight;
an astringent, preferably aluminum chloride, intended to help stop bleeding, resulting in good surface hemostasis.

This patent led to the development, and then the marketing of the product known as Expasyl® which opened up a new route of treatment in all indications of temporary sulcular opening.

This product is in fact particularly advantageous, in particular in comparison with the materials that can be hardened by chemical reaction, owing to its simplicity of use (absence of premixing) and its rapid action (no hardening time).

In addition, since it contains aluminum chloride, the Expasyl® product results in opening of the sulcus with excellent hemostasis.

However, the Expasyl® product has a certain number of drawbacks.

First of all, owing to the very hydrophilic and water-soluble nature of kaolin clay, this product must be kept away from saliva in order to avoid premature dilution thereof, thus creating a situation of discomfort for the patient, who must avoid closing the mouth during its use.

For the same reasons, once put in place, this product cannot be used together with an instrument comprising a water spray.

Whatever the precautions taken, it has been noted that the Expasyl® product is diluted with saliva and sulcular fluids, losing its consistency and its effectiveness after approximately two minutes, limiting accordingly the working time for the dentist.

Moreover, removal of this product after use takes a relatively long time and requires a large amount of water, in particular in order to avoid any deposit of kaolin on the dentine, which can interfere for example with bonding, and also in order to remove the chloride which can interfere with the setting of impression materials of the polyether category.

The use of a large amount of water leads to disintegration of the product and the release of its constituent components, and in particular of the aluminum chloride. As it happens, aluminum chloride has a particularly unpleasant and persistent taste and can lead to irritation of the mucous membranes.

Finally, due to its consistency, this product can only be extruded with a specific instrument designed to multiply the manual force applied.

A material that can be used to form a dressing on the oral mucous membranes or on the skin, consisting of a biocompatible paste containing, as essential constituent, natural kaolin, a humectant and a hydrogel-forming agent, preferably colloidal silica, is also known through document WO 2007/128926. Such a material is currently marketed under the trade name Hémostasil® and has hemostatic properties.

This material contains between 35 and 55% by weight of kaolin and disintegrates very rapidly in the presence of water. As a result, it has the same drawbacks as the Expasyl® product.

Furthermore, due to its consistency, this material does not allow opening of the sulcus or maintaining an opening obtained by gingivectomy. It cannot therefore be used in the applications envisioned by the present invention.

It also should be noted that kaolin used in a relatively large proportion in this product forms a fluid paste after hydration, which changes consistency under the effect of the pressure exerted for extrusion. Thus, the hardness of this paste increases with the pressure exerted as well as with the speed of extrusion.

In this context, the objective of the present invention is to solve the technical problem consisting of the provision of a novel hemostatic compound:
which can be easily extruded, notably by simple manual force, without changing consistency,
which allows maintaining the sulcular opening after a gingivectomy while guaranteeing that an excellent hemostasis is obtained, and
which has sufficient cohesion in aqueous medium to avoid losing its mechanical properties for a duration compatible with any type of treatment and can be removed in a single piece by an air and/or water spray, or using an appropriate instrument generally designated by the term “probe”.

Thus, according to a first aspect, the subject of the present invention is a hemostatic composition in the form of an extrudable hydrophilic paste, characterized in that it contains, expressed as percentage by weight relative to the total weight of said composition:
between 5 and 15% of an astringent, preferably aluminum chloride;
between 2 and 15% of kaolinitic clay, preferably kaolin;
between 10 and 20% of a texturizing agent comprising one (or more) naturally-occurring polysaccharide(s) with gelling or thickening power optionally combined with plant fibers;
between 50 and 70% of water;
between 0 and 20% of a humectant;
   the total quantity of water and humectant being comprised between 55 and 75% by weight relative to the total weight of the composition.

As is understood, the originality of the composition in accordance with the present invention lies in the nature and the proportions of the various ingredients of which it is composed, particularly the texturizing agent.

This texturizing agent comprises one (or more) natural polysaccharide(s) with gelling or thickening power optionally combined with plant fibers and represents between 10 and 20%, preferably between 15 and 20% by weight, relative to the total weight of the material.

Naturally occurring polysaccharides with a gelling or thickening power that can be used in the context of the present invention are advantageously chosen from the group made up of pectins; galactomannans of guar, locust bean or tara seeds; scleroglucan; xanthan; carrageenans, and mixtures thereof and are preferably food quality.

In combination with the kaolin clay, they allow obtaining a composition having rheological properties that are stable over time. In particular, they form a hydrophilic matrix insensitive to pressure, so that, unlike the products of the prior art, the composition keeps its consistency under the effect of the pressure exerted for its extrusion.

To this effect, the texturizing agent in accordance with the invention is free of or contains only a small amount, less than 10% by weight preferably less than 5% by weight, and more preferably less than 1% by weight, relative to the weight of the texturizing agent, of alginates. Indeed, it has been observed that gels that can be formed from alginates are irreversible and do not exhibit a consistency stable over time that is compatible with their use in the context of the invention.

According to a preferred embodiment of the invention, the texturizing agent, and therefore the composition of the invention are free of alginates.

Preferably, the polysaccharides will be chosen from carrageenans.

Carrageenans are generally obtained by hot extraction of red algae of the family Rhodophyceae, in particular of Chondrus and Eucheuma.

They are copolymers of D-galactose ether sulfate and of 3,6-anhydro-D-galactose which can be in the form of salts, in particular potassium or calcium salts.

Advantageously, the carrageenans used in the context of the invention are of iota type, such as, in particular, the products sold by the company FMC BIOPOLYMERE under the names Viscarin® or Gelcarin®.

Excellent results have been obtained in the context of the present invention with the product Viscarin® PC 389.

The texturizing agent may be exclusively made up of one or more polysaccharides but can also comprise plant fibers.

The plant fibers that can be used in the context of the present invention are advantageously chosen from pea fibers and oat fibers.

Such products are well known in the cosmetics field and are, for example, sold by the company Beacon CMP under the name Tech-O®.

Generally, the weight ratio of the plant fibers to the polysaccharides is comprised between 1:10 and 1:50.

The composition according to the invention comprises between 2 and 15%, preferably between 3 and 10%, by weight of kaolin clay.

Beyond this proportion, the composition obtained has been observed to change consistency under the effect of the pressure applied while it is being used.

Advantageously, the kaolin clay is made up of natural kaolin containing at least 80%, preferably at least 90%, and more preferably at least 95% by weight of kaolinite.

The astringent that can be used in the context of the present invention is generally chosen from the group consisting of iron or aluminum chlorides and sulfates, potassium aluminum sulfate, and mixtures thereof.

In the currently preferred embodiment of the invention, the astringent is aluminum chloride and can be used in an amount between 2 and 10% by weight, advantageously between 7 and 10% by weight, relative to the total weight of the insertion material.

The amount of water contained in the composition in accordance with the invention can vary, in particular according to the nature of the texturizing agent and to whether or not it is combined with a humectant.

Water, preferably purified water, is used in an amount sufficient to obtain a cohesive paste that can be easily extruded, preferably using a conventional syringe with simple manual force, without needing a device for multiplying manual force.

Generally, the water is present in the composition in an amount of between 50 and 70% by weight, preferably between 55% and 65% by weight, relative to the total weight of the composition.

The humectant that can be used in the context of the present invention is generally chosen from the group consisting of glycerol, polyethylene glycol and sorbitol, and mixtures thereof. It will preferably be glycerol (or glycine).

The humectant does not constitute an essential component of the composition according to the invention. It is advantageously used to improve the stability of the composition, in particular to prevent this composition from drying out during storage thereof.

When it is used, the humectant is present in the composition in an amount of less than 20% by weight, preferably between 8 and 16% by weight, relative to the total weight of the material.

A currently preferred hemostatic composition, in the context of the present invention, comprises:
between 7 and 10% of an astringent, preferably aluminum chloride;
between 3 and 10% of kaolinitic clay, preferably kaolin;
between 15 and 20% of a texturizing agent;
between 55 and 65% of water;
between 8 and 15% of a humectant;

The composition in accordance with the present invention can also comprise up to 1% by weight, and preferably from 0.01 to 0.5% by weight of at least one additive chosen from dyes and flavorings, preferably of food grade.

Any type of dye can be used in the context of the present invention.

As regards dental applications, a dye that has a color which stands out clearly against the color of the tooth and of the gum, for instance blue, yellow or green, will preferably be used.

The dye can be used in an amount of generally between 0.005% and 0.05%, preferably of approximately 0.015% by weight, relative to the total weight of the material.

There is also no limitation as to the nature of the flavoring that can be used in the context of the present invention. It may be an almond, mint or lemon flavoring, for example.

The amount of flavoring that can be used is generally between 0.1 and 1%, and preferably approximately 0.5% by weight, relative to the total weight of the material.

The composition in accordance with the invention may also comprise up to 10% by weight, and preferably from 2 to 10% by weight, of at least one active substance chosen from local anesthetics.

Among the local anesthetics that can be used in the context of the invention, mention may be made of lidocaine, prilocaine, bupivacaine, benzoin, tetracaine, mepivacaine and ropivacaine.

Advantageously, the local anesthetic is lidocaine and can be used in an amount of approximately 2 to 7% by weight, relative to the total weight of the composition.

According to a second aspect, the present invention concerns a hemostatic composition such as previously described, for its use as a product intended to treat hemostasis and maintain sulcular opening after a gingival eviction procedure.

The composition in accordance with the present invention has original physicochemical properties which allow it to meet the requirements of its use in the dental field, in particular to ensure good hemostasis and maintain the sulcular opening after a gingival eviction procedure.

Firstly, this composition has a dissolution profile in aqueous medium that allows prolonged diffusion of the astringent agent in the mouth, sufficiently long to ensure good hemostasis even in the event of major bleeding such as occurs after a gingival eviction procedure.

This dissolution profile has been assessed by measuring release of the astringent agent over time.

More specifically, the amount of astringent released, measured according to the dissolution method described in the European Pharmacopoeia, 6th edition (Ph. Eur. 2.9.3 (01/2008)) at 150 rpm in 700 ml of purified water at 37° C., is:
less than 65% after 15 min;
less than 75% after 30 min;
less than 85% after 60 min.

The amount of astringent released can be determined by assaying methods well known to those skilled in the art.

In the case of aluminum chloride, which constitutes the astringent currently preferred, this assay can be carried out by complexometric titration, for example according to the method described in the European Pharmacopoeia, 6th edition (01/2008) in chapter 2.5.11.

It has been shown that the composition in accordance with the present invention releases the astringent much more slowly than the Expasyl® product, and therefore allows prolonged use in the mouth.

By way of example, the amount of astringent released after 15 min with the Expasyl® product is approximately 77%, whereas this same amount is less than 65% with a material according to the invention.

Moreover, the insertion material in accordance with the present invention exhibits excellent cohesion over time. Thus, this composition shows no visible disintegration after 1 h according to the method for disintegration of tablets and capsules described in the European Pharmacopoeia, 6th edition (01/2008) in chapter 2.9.1, for a sample having a diameter of 10 mm and a thickness of 4 mm and a disintegration medium consisting of purified water at 37° C.

In the same test, it was noted that the Expasyl® material disintegrates completely after 15 min.

Finally, extrusion tests have shown that the composition according to the invention can be extruded with simple manual force.

Thus, the pressure necessary to extrude a bead of 2.2 mm diameter at a rate of 1 mm/s, from a cylindrical cartridge of 30 mm long and 4.5 mm inner diameter is less than 100 N/cm², preferably around 50 N/cm².

The composition in accordance with the present invention can be readily produced by simple mixing of its constituents.

Generally, a composition according to the invention can be obtained:
by preparing a mixture containing purified water and the astringent and, optionally, a humectant such as glycerol and a dye such as patent blue; and
by adding to the solution thus prepared a texturizing agent such as a polysaccharide, preferably of iota-carrageenan type, optionally combined with a plant fiber and kaolin clay.

The invention will be understood more clearly on reading the following nonlimiting examples.

### EXAMPLE 1

### Step 1

In a 20-l beaker made of low-density polyethylene, 10.757 liters of water purified according to the European Pharmacopoeia 6.3 standard (01/2009:0008) and 3 kg of aluminum chloride hexahydrate (Panreac) were mixed and stirred for 10 minutes using a pneumatic stirrer with a turbine. Owing to the exothermicity of the reaction between the water and the aluminum chloride, all necessary precautions known to those skilled in the art must be used.

Then 2.6 g of patent blue (Spectracol) were gradually added with stirring into the vortex of the abovementioned mixture, and the resulting mixture was stirred for 5 minutes using the pneumatic stirrer until the solution was homogeneous.

Still with stirring, 2 kg of glycerol (La Cooper) were then added into the vortex of the mixture, and the stirring was maintained for a further 10 minutes using the pneumatic stirrer.

### Step 2

All of the mixture prepared in step 1, 3.1 kg of carrageenans of Viscarin® PC 389 type and 1.14 kg kaolin clay were introduced into the tank of a Z arm blender equipped with a cooling system (circulation of cold water in the double jacket), and then pre-mixed at a speed of 19 rpm for 20 minutes.

The blender can be optionally stopped so as to detach the paste from the walls of the blender.

The resulting mixture is then stirred for a further period of 90 minutes at a speed of 19 rpm, thus producing a composition according to the invention.

### EXAMPLES 2 AND 3

Various hemostatic compositions according to the invention were prepared by following a process analogous to that described in example 1. The composition of each of the materials used is given as percentage by weight relative to the total weight of the composition in the following Table I.

TABLE 1

| | Example 1 | Example 2 | Example 3 |
|---|---|---|---|
| Water | 60.537 | 64.737 | 64.737 |
| Kaolin | 5.7 | 3.5 | 3.5 |
| AlCl₃ | 8.25 | 8.25 | 8.25 |
| Glycerol | 10 | 10 | 10 |
| Carrageenan⁽¹⁾ | 15.5 | | 3.5 |
| Oat fiber | | 13.5 | 10 |
| Pea fiber | 0.013 | 0.013 | 0.013 |
| Dye | 100 | 100 | 100 |

⁽¹⁾Viscarin ® PC 389 sold by the company FMC BIOPOLYMERE.

### EXAMPLE 4

### Demonstration of the Physicochemical Properties of the Composition According to the Invention

Dissolution Tests
Protocol used:
Protocol followed: European Pharmacopoeia 2.9.3 “Dissolution test for solid dosage forms”
Type of device used: Sotax paddle apparatus
Paddle rotational speed: 150 rpm
Thermostated bath temperature: 37° C.
Tank volume=700 ml
Dissolution medium sampling mode:
   time: t=15 min, 30 min and 60 min
      Method: samples taken using a graduated glass pipette of class A+ precision
      Sample volume=50 ml
Method of analysis: Complexometric titration (according to European Pharmacopoeia 2.5.11)

Results Obtained:

The dissolution results obtained with the composition of Example 1 were compared with those obtained with the Expasyl® product. These results, which have been reported in Table II, show very clearly that the compositions according to the invention have a dissolution rate characterized by a significantly slower release of the astringent than in the case of Expasyl®.

TABLE II

| | Time (min) | | |
|---|---|---|---|
| | 15 | 30 | 60 |
| Expasyl | 77.53 | 88.35 | 94.90 |
| Example 1 | 61.77 | 70.95 | 75.72 |

Disintegration Test
tests carried out according to the European Pharmacopoeia 2.9.1 (disintegration of tablets and capsules); test A—tablets and capsules of normal sizes with use of cylindrical disks
immersion temperature: 37° C.
immersion medium: water purified according to the European Pharmacopoeia 6.3 standard (01/2009:0008)
sample diameter: 10 mm
sample thickness: 4 mm
disintegration resistance: 1 hour

Extrusion Tests

The extrusion tests were carried out using a cylindrically shaped cartridge, 30 mm long and 4.5 mm inner diameter, furnished at one of its ends with a plunger equipped with an O ring, and having a conical convergent shape at the other end of an angle of 45° emerging into an opening of 2.2 mm inner diameter.

The force necessary to extrude the composition in accordance with the invention was measured by means of a texture analyzer (sold under the trade name Stable Microsystem by Swantech International), with an applied plunger speed of 1 mm/s.

This analyzer comprises a plate onto which is attached an arm provided with a device capable of linearly moving a mobile part with an adjustable course and speed. The force applied is continuously measured and recorded as a curve with the distance traveled by the mobile part expressed in millimeters on the x-axis and the force exerted expressed in grams on the y-axis. The mobile part travels perpendicularly to the table.

The cartridge is placed between the table and the mobile part of the analyzer, the end with the plunger being connected to the mobile part of the analyzer.

The mobile part was activated at a rate of 1 mm/s along a trajectory of 30 mm.

The force was recorded after a threshold of 50 g.

Under these conditions, it has been observed that the force necessary to extrude the composition according to Example 1 has a maximum at the beginning of extrusion of around 800 g. Since the area of the plunger is 0.1589 cm², the calculated pressure was therefore around 50 N/cm².

Under the same conditions, the pressure necessary to extrude the Expasyl® product is around 300 N/cm².

This test shows that, unlike the Expasyl® product, the composition in accordance with the present invention can be extruded with simple manual force, without requiring a manual force multiplying device.

This composition, unlike the Expasyl® product, may therefore be packaged in a conventional syringe and therefore does not require a specific device for its extrusion.

The hemostatic composition according to the invention therefore has numerous advantages:
it may be extruded by simple manual force and does not require any special device for use;
its consistency is independent of the pressure applied and remains constant during its extrusion;
it may be extruded in the form of a bead with good cohesion in aqueous medium and therefore allows maintaining the sulcular opening, notably after a gingivectomy, or even widening the gingival crevice in the case of a thin gingiva;
it allows slow release of the astringent;
it allows excellent hemostasis, even in the event of major bleeding;
it may be eliminated in one piece with an air and/or water spray, or a suitable instrument.

1. Hemostatic composition in the form of an extrudable hydrophilic paste, characterized in that it contains, expressed as percentage by weight relative to the total weight of said composition:
between 5 and 15% of an astringent, preferably aluminum chloride;
between 2 and 15% of kaolinitic clay, preferably kaolin;
between 10 and 20% of a texturizing agent comprising one (or more) naturally-occurring polysaccharide(s) with gelling or thickening power optionally combined with plant fibers;
between 50 and 70% of water;
between 0 and 20% of a humectant;
the total quantity of water and humectant being comprised between 55 and 75% by weight relative to the total weight of the composition.

2. Hemostatic composition according to claim 1, which comprises between 7 and 10% of an astringent.

3. Hemostatic composition according to claim 1, which comprises between 3 and 10% of a kaolinitic clay, preferably kaolin.

4. Hemostatic composition according to claim 1, which comprises between 15 and 20% of a texturizing agent.

5. Hemostatic composition according to claim 1, which comprises:
between 7 and 10% of an astringent, preferably aluminum chloride;
between 3 and 10% of kaolinitic clay, preferably kaolin;
between 15 and 20% of a texturizing agent;
between 55 and 65% of water;
between 8 and 15% of a humectant.

6. Hemostatic composition according to claim 1, wherein the astringent is chosen from the group consisting of iron or aluminum chlorides and sulfates, potassium aluminum sulfate, and mixtures thereof.

7. Hemostatic composition according to claim 1, wherein the kaolinitic clay is made up of natural kaolin containing at least 80%, preferably at least 98% by weight of kaolinite.

8. Hemostatic composition according to claim 1, wherein the above-mentioned naturally-occurring polysaccharide is advantageously chosen from the group made up of pectins; galactomannans of guar, locust bean or tara seeds; scleroglucan; xanthan and carrageenans, preferably iota, and mixtures thereof.

9. Hemostatic composition according to claim 1, wherein the above-mentioned plant fibers are chosen from pea fibers and oat fibers.

10. Hemostatic composition according to claim 1, wherein the above-mentioned astringent is aluminum chloride.

11. Hemostatic composition according to claim 1, wherein the above-mentioned humectant is chosen from the group consisting of glycerol, polyethylene glycol and sorbitol, and mixtures thereof.

12. Hemostatic composition according to claim 11, wherein the above-mentioned humectant is glycerol.

13. Hemostatic composition according to claim 1, which further contains up to 1% by weight, and preferably from 0.01 to 0.5% by weight of at least one additive chosen from dyes and flavorings, preferably of food grade.

14. Hemostatic composition according to claim 1, which further contains up to 10% by weight, and preferably from 2 to 10% by weight of at least one additive chosen from local anesthetics, preferably lidocaine.

15. Hemostatic composition according to claim 1, which may be extruded as a bead of 2.2 mm diameter at a rate of 1 mm/s, from a cylindrical cartridge of 30 mm long and 4.5 mm inner diameter by applying a pressure less than 100 N/cm², preferably less than 50 N/cm².

16. Hemostatic composition according to claim 1, for its use as a product intended to treat hemostasis and maintain sulcular opening after a gingival eviction procedure.
